(19) United States
(12) Patent Application Publication
(10) Pub. No.: US 2020/0268399 A1
(43) Pub. Date: Aug. 27, 2020

(54) HAEMOSTATIC DEVICE

(71) Applicant: Christopher Hill, Cottesloe (AU)

(72) Inventor: Christopher Hill, Cottesloe (AU)

(21) Appl. No.: 16/063,792

(22) PCT Filed: Dec. 21, 2016
(86) PCT No.: PCT/IB2016/057848

§ 371 Date: Jun. 19, 2018

(30) Foreign Application Priority Data
Dec. 24, 2015 (NZ) 715600

(51) Int. Cl. A61B 17/26

(52) U.S. Cl. CPC A61B 17/26; A61B 2017/12004

(57) ABSTRACT

The invention provides a haemostatic device adapted to apply direct pressure to alleviate post-operative haemorrhage, for example post tonsillectomy. The device comprises an anchoring structure configured to engage with a patient's teeth, and an arm mounted on the anchor with a wound contacting portion at an end of the arm.

### FIELD OF THE INVENTION

The present invention relates to methods and devices for promoting, managing and/or maintaining haemostasis. In particular but not exclusively, the present invention relates to haemostatic devices and methods for management of post-tonsillectomy haemorrhage.

### BACKGROUND TO THE INVENTION

Post-tonsillectomy haemorrhage is a relatively common complication of tonsillectomy procedures. Haemorrhage can range from minor to life-threatening. In major cases, patients typically require emergent otolaryngology review and surgical intervention. These services may be “off-site”, requiring patient transfer to a different facility.

Current methods of managing post-tonsillectomy haemorrhage in the emergency setting include: hydrogen peroxide gargles, spraying of vasoconstrictive medications to the tonsillar fossa, and direct pressure on the tonsillar fossa with gauze held in forceps.

Application of direct pressure is an effective way of managing haemorrhage. However, this technique is often poorly tolerated by the patient due to pain and the gag reflex. Adequate and sustained pressure is difficult to achieve due to patient compliance. It requires an experienced operator to administer and continuously maintain this treatment until haemostasis is achieved or definitive otolaryngology management is at hand. This can have a significant impact on resources, especially in cases where the patient requires transfer to a different medical facility.

Accordingly, it is an object of the present invention to provide devices and methods for managing haemorrhage within the oral cavity, in particular post-tonsillectomy haemorrhage, which address at least some of the issues above, or at least to provide the public with a useful choice.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### SUMMARY OF THE INVENTION

According to one aspect, the invention broadly comprises a haemostatic device comprising:
at least one anchoring structure configured to be reversibly engaged with a patient's teeth,
an arm connected to said anchoring structure, and
at least one wound-contacting portion at or toward an end of the arm.

According to another aspect the device further comprises a user-manipulation portion at or toward the other end of said arm, said user-manipulation portion being manipulatable to adjust:
a) positioning of said wound-contacting portion, and
b) pressure applied by said wound-contacting portion(s) onto one or more wound(s).

According to another aspect the invention comprises the use of a device according to the previous clauses, for haemostasis of a patient's tonsillar fossa following a tonsillectomy.

According to another aspect the anchoring structure comprises at least one bite block to be held between the patient's mandibular and maxillary teeth.

According to another aspect said wound-contacting portion is supported from said anchoring structure via the elongate arm.

According to another aspect said elongate arm is integrally formed with said user-manipulation portion.

According to another aspect said connection between said arm and said anchoring structure is moveble, to adjust one or more of:
a. positioning of said wound-contacting portion,
b. pressure applied by said wound-contacting portion onto said wound,
c. shape and configuration of said wound-contacting portion,
d. distance between said wound-contacting portion and said user-manipulation portion.

According to another aspect the movable connection is resiliently deformable via one or more of:
a. a spring or other biasing structure,
b. inherent resilience of the material of said portion.

According to another aspect said moveable connection is plastically deformable, such that said portion may be shaped prior to application of said wound-contacting portion on said wound.

According to another aspect at least part of said user-manipulation portion comprises at least one deformable portion.

According to another aspect a connection between said wound-contacting portion and said arm is moveable or deformable.

According to another aspect a connection between said anchoring structure and said arm is deformable.

According to another aspect a connection between said anchoring structure and said user-manipulation portion is deformable.

According to another aspect the haemostatic device of any one of the previous clauses, comprising at least one portion which is movable relative to one or more other portions, to adjust one or more of:
a. positioning of said wound-contacting portion,
b. pressure applied by said wound-contacting portion onto said wound,
c. distance between said wound-contacting portion and said user-manipulation portion.

According to another aspect said at least one portion is reversibly movable relative to said one or more other portions.

According to another aspect said anchoring structure is slidably movable along said arm.

According to another aspect said anchoring structure is rotatably movable relative to said arm.

According to another aspect said wound-contacting portion and said arm are rotatably movable and/or pivotable relative to each other.

According to another aspect said arm is pivotably supported on said anchoring structure.

According to another aspect said wound-contacting portion is slidably or telescopically movable along the arm.

According to another aspect the invention comprises the haemostatic device of any one of the preceding clauses, wherein the user-manipulation portion comprises an elongate handle.

According to another aspect the wound-contacting portion comprises a pad.

According to another aspect said pad comprises a compressible material to conform to the shape and/or configuration of said wound.

According to another aspect the invention comprises the haemostatic device of any one of the previous clauses, wherein said pad is one or more of:
a. compressible,
b. porous,
a. elastic,
b. spongy,
c. absorbant,
d. inflatable/expandable.

According to another aspect said pad comprises one or more of:
a. woven fabric,
b. non-woven fabric,
c. foam,
d. gauze,
e. sponge,
f. fibre,
g. paper,
h. a gel block, or a gel filled capsule,
i. plastic, or
j. rubber.

According to another aspect said wound-contacting portion comprises one or more pre-applied:
a. Haemostatic agent,
b. Antiseptic agent,
c. Anesthetic agent,
d. Antibiotic agent,
e. Anti-inflammatory agent,
f. Cleansing agent,
g. Irrigation agent,
h. Analgesic agent,
   said one or more agents deliverable to said wound when said wound-contacting portion is applied to said wound.

According to another aspect said wound-contacting portion comprises at least one reservoir containing said one or more agent(s).

According to another aspect said wound-contacting portion comprises an inflatable balloon, and wherein said device further comprising means of applying a pressure to inflate said balloon.

According to another aspect the device further comprising one or more conduits extending along a length of the device, configured for one or more of:
a. applying suction,
b. dispensing one or more medicaments,
c. dispensing irrigation solution,
d. allowing passage of one or more medical devices,
e. applying pressure to inflate a balloon of said wound-contacting portion,
f. providing illumination.

According to another aspect said wound-contacting portion is removably attached to said arm.

According to another aspect said anchoring structure is removably attached to the device.

According to another aspect said wound-contacting portion is substantially spherical or discoid or ovoid or cylindrical in shape, and wherein a largest length measurement of said wound-contacting portion is less than approximately 50 mm.

According to another aspect the device, comprising two said wound-contacting portions, spaced from each other and configured to be applied one to a wound on each of a patient's tonsillar fossa following a bilateral tonsillectomy.

According to another aspect said user-manipulation portion is between 50 mm and 200 mm in length, and configured to be gripped and manipulated by a patient's hand.

According to another aspect said user-manipulation portion is configured to be manipulated by a patients tongue.

According to another aspect said elongate arm extends substantially parallel to said anchoring structure, such that said elongate arm extends in use along a peripheral portion of the patient's mouth.

According to another aspect the elongate arm extends in use substantially parallel to the alveolar process on one or both lateral sides of the patient's mouth.

According to another aspect said anchoring structure comprises a channel sized and shaped to fit over a patient's teeth.

According to another aspect said anchoring structure includes a deformable/mouldable material, to improve and/or maximise dental contact.

According to another aspect said anchoring structure comprises a channel sized and shaped to fit over a patient's teeth and said deformable/mouldable material is located in the channel.

According to another aspect the invention broadly comprises a haemostatic device comprising:
an arm, and
at least one wound-contacting portion at or toward an end of the arm.

According to another aspect the invention broadly comprises a method of controlling bleeding of a patient from one or more wound(s) using the haemostatic device of any one of claims 1 to 40, comprising:
a. engaging said anchoring structure to the patient's teeth,
b. positioning said wound-contacting portion on said wound.

According to another aspect the invention comprises a method of controlling bleeding of a patient from one or more wound(s) using the haemostatic device of any one of the previous clauses, further comprising a step of manipulating said user-manipulation portion to adjust the pressure applied by said wound-contacting portion onto said wound.

According to another aspect the patient manipulates said user-manipulation portion to adjust one or both of:
a. position of said wound-contacting portion,
b. pressure applied by said wound-contacting portion onto said wound.

According to another aspect the invention broadly comprises a haemostatic device, substantially as herein described, and with reference to any one or more of the drawings.

According to another aspect the invention broadly comprises a method of controlling bleeding of a patient from one or more wound(s), substantially as herein described, and with reference to any one or more of the drawings.

The term “comprising” as used in this specification and claims means “consisting at least in part of”. When interpreting each statement in this specification and claims that includes the term “comprising”, features other than that or those prefaced by the term may also be present. Related terms such as “comprise” and “comprises” are to be interpreted in the same manner.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

The invention consists in the foregoing and also envisages constructions of which the following gives examples only.

### BRIEF DESCRIPTION OF THE DRAWINGS

Preferred embodiments of the invention will be described by way of example only and with reference to the drawings, in which:

FIG. 1 is a perspective view of a haemostatic device according to one embodiment,

FIG. 2 is a plan view of the haemostatic device of FIG. 1, illustrated in position within a patient's mouth,

FIG. 3 is a perspective view of a haemostatic device according to another configuration, illustrated in position within a patient's mouth,

FIGS. 4a and 4b show further alternative configuration of the haemostatic device,

FIG. 5 shows another configuration of the haemostatic device, and in particular a connection between the bite block and elongate arm,

FIG. 6 shows another alternative connection between the bite block and elongate arm,

FIG. 7 shows another alternative connection between the bite block and elongate arm,

FIG. 8 shows another configuration of the haemostatic device.

FIG. 9 shows another configuration of the haemostatic device, with pivoting and/or removable wound contacting pad.

FIG. 10 shows yet another configuration of the wound contacting pad,

FIG. 11 shows various configurations of the bite block.

### DETAILED DESCRIPTION OF PREFERRED EMBODIMENTS

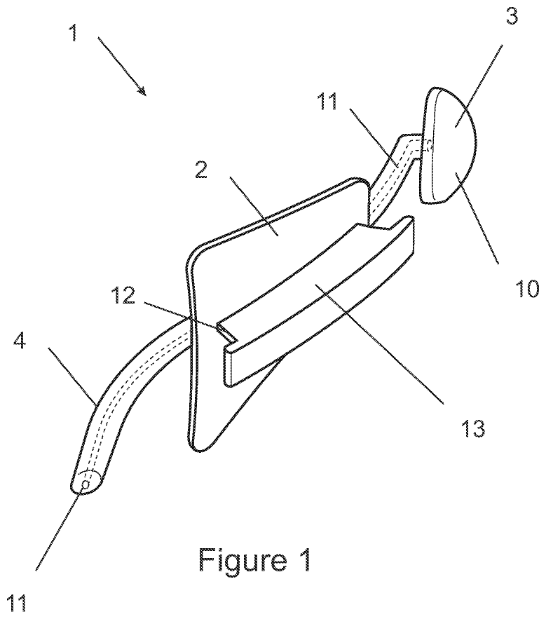

FIG. 1 shows one embodiment of the haemostatic device 1 comprising at least one anchoring structure 2 which is configured to be reversibly engaged with a patient's teeth, and at least one wound-contacting portion 3 which is supported on the anchoring structure 2, (via an arm) and is configured to contact and apply pressure on the patient's wound(s).

The device 1 may further comprise a user-manipulation portion 4 which is graspable by a patient. Where present, the user-manipulation portion 4 may be manipulated by the patient or a medical operator to adjust the position of the wound-contacting portion 3 on/in the patient, and further to adjust the pressure applied by the wound-contacting portion 3 onto the wound.

In other embodiments, the haemostatic device 1 may not include a user-manipulation portion 4. In this case, the position of the wound-contacting portion 3 and the pressure applied by the wound-contacting portion 3 may be adjusted by shaping the device 1 prior to insertion into the patient's mouth and/or by pushing or pulling directly on the wound-contacting portion 3 once the device 1 has been anchored on/against the patient's teeth.

The haemostatic device shown in FIG. 1 is particularly suited for managing bleeding of the patient's tonsillar fossa after a tonsillectomy. In particular, the present device may be operated by the patient independently, with minimal medical operator intervention.

As discussed, application of direct pressure onto the wounded regions is an effective method of managing haemorrhage. However, the tonsillar fossa is difficult to access, let alone access for a prolonged period of time in order to maintain sufficient pressure on the wounded region. It is expected that it may typically be necessary to apply pressure to the wound for at least 10 minutes, before the wound could be left exposed, and monitored to further bleeding. However, if bleeding is moderate or severe, and does not stop after 10 minutes or more, then further pressure could be applied until surgical management is available. In more severe cases, it is anticipated that prolonged pressure (e.g. for up to an hour or more) may be required, and it would not be expected to cause undue complications.

The prior art method of applying pressure via a gauze held in forceps requires ongoing medical operator intervention, and is often poorly tolerated by the patient, due to the intrusion, pain and gag reflex.

In contrast, the present device facilitates the application of consistent, direct pressure on the tonsillar fossa for extended periods of time. In preferred configurations, the device allows the patient to operate the device and manage the haemostatic treatment on their own, with little or no intervention from a medical operator once the device is in place. In further preferred configurations, the device allows for adjustment of the position and pressure applied to the wound, prior to and during treatment, for more effective treatment, as will be discussed in more detail below.

The anchoring structure 2 secures the device 1 to the patient, such that once the device is in place, it may be unnecessary to hold on to the device externally (i.e., reducing the need for ongoing medical operator intervention). In particular, where the anchoring structure is a bite block, pressure from the teeth biting together, will firmly secure the device.

The wound-contacting portion 3 is preferably supported on the anchoring structure 2 via an elongate arm 7, such that the wound-contacting portion 3 extends from the anchoring structure 2 and reaches the patient's tonsillar fossa 6. It is preferred that the elongate arm 7 traces a peripheral pathway (for example along an inner margin of the teeth), in order to minimise discomfort.

In embodiments of the device 1 that include the user-manipulation portion 4, the user-manipulation portion 4 may be integrally formed with the arm 7, and may also include a handle to improve grip etc.

A user-manipulation portion 4 may provide a secondary stabilising structure for the patient to grip, in addition to the secure connection already provided by the anchoring structure 2 on the patient's teeth. The user-manipulation portion 4 may comprise an elongate handle, configured to be gripped and manipulated by the patient's or medical operator's hand. In some examples, the user-manipulation portion may be between 50 mm and 200 mm in length, such that a substantial portion of the user-manipulation portion can extend out of the patient's mouth in use.

In other configurations, the user-manipulation portion 4 may be configured to be manipulated by the patient's tongue. In some cases, the user-manipulation portion 4 may be (at least part of) the elongate arm 7 or other connection between the anchoring structure 2 and the wound-contacting portion 3, which the patient can move with their tongue in order to adjust the position of the wound-contacting portion or the pressure applied by the wound-contacting portion on the wound.

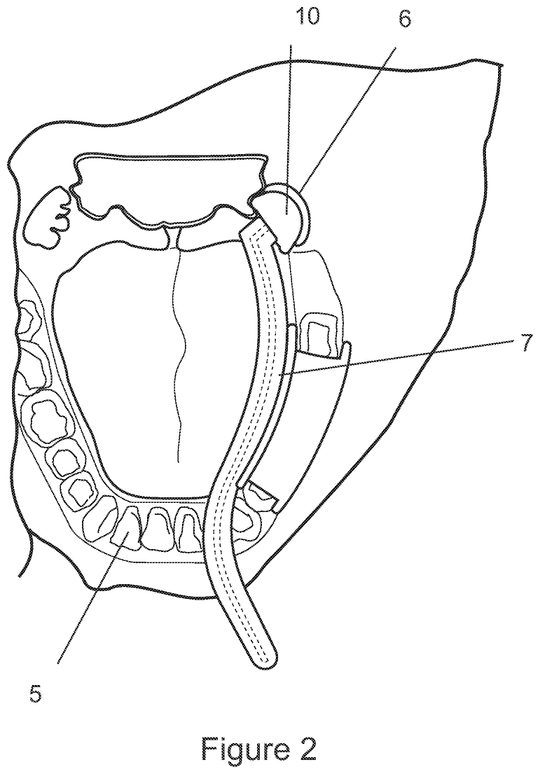

In some configurations, as shown in FIGS. 1 and 2, the anchoring structure 2 may comprise a body including one or more recesses shaped to receive a patient's teeth. For example, the block may be configured as a channel or bracket 12 that may be attached onto/over a row of the patient's teeth 5.

In one example, as shown in FIG. 2, the anchoring structure 2 is attached over several of the patient's mandibular teeth.

It will be appreciated that the engagement of the anchoring portion 2 over a patient's teeth may allow some relative movement between the bite block and the patient's teeth. Allowing some movement may be useful for positioning the device properly to achieve pressure on the patient's tonsillar fossa. In alternative configurations, the anchoring of the anchoring structure 2 to a patient's teeth, may be approximately rigid.

It is preferred that the anchoring structure is capable of it least retaining its position on a patient's teeth without the need for a patient to bite down. This configuration allows the patient to have their mouth open during the treatment, which would facilitate positioning of the wound-contacting portion onto the wound, and additionally allow observation of the wound (e.g., by the medical operator) during the treatment. However, is also preferred that the structure allows a patient to bite down on it, to further grip the anchoring structure firmly between the teeth, to provide a more secure anchoring.

In order to improve grip between the teeth and bite block, the bite block surface may be textured to provide improved friction, in order to reduce sliding and/or rotation of the block with respect to the teeth.

It should be appreciated that the anchoring structure 2 could be additionally or alternatively configured to be attached to one or more of the patient's maxillary teeth, and on one or both sides of the patient's mouth.

In another configuration, as shown in FIG. 3, the anchoring structure 2 may comprise a bite block 13 configured to be gripped between the patient's mandibular and maxillary teeth 5 and/or jaw bones, and may include a deformable and/or mouldable and/or displaceable material that at least partially moulds to the patient's teeth providing a more secure fit (not shown). For example, the channel 12 maybe partially (or completely) filled with a deformable type material.

Further, the contact portion of the bite block (i.e. the surface that is bitten on) may be crosshatched/ridged or otherwise textured, to provide sufficient friction against the teeth to reduce sliding and/or rotation of the block with respect to the teeth. In addition to, or alternatively, the block 13 may be surfaced with the deformable/mouldable material, to improve and/or maximise dental contact in order to provide additional stability.

It is preferred in some configurations that the material properties of the deformable/mouldable material, are such to allow adjustment and/or repositioning, in a way that the material will remould to the new configuration repeatedly.

In other alternative configurations, a more permanent moulding material may be used that results in creation of a permanent dental recess in the block. However, it will be appreciated that in order to be practical, the material will need to be cured relatively quickly and easily, without bonding to the teeth. It is anticipated that material such as UV cured polymers, may be suitable, but many other suitable materials could also be employed.

One or more portions or parts of the device 1 may be further deformable and/or moveable to adjust the positioning of the wound-contacting portion 3 relative to the wound, and/or the pressure applied by the wound-contacting portion 3 onto the wound, and/or the distance between the wound-contacting portion 3 and the user-manipulation portion 4. Additionally or alternatively, the shape and configuration of the wound-contacting portion may also be adjusted and conformed to the patient's specific anatomy by deforming the wound-contacting portion 3.

In some configurations, the position of the wound-contacting portion 3 relative to the anchoring structure 2 may be adjustable. In this way, the position of the wound-contacting portion 3 and/or the pressure that the wound-contacting portion applies onto the wound may be adjusted and customised according to the patient's specific anatomy. For example, the elongate arm 7 may be deformable, such that it may be shaped to suit the patient's anatomical structure before or during positioning of the device 1 in the patient's mouth.

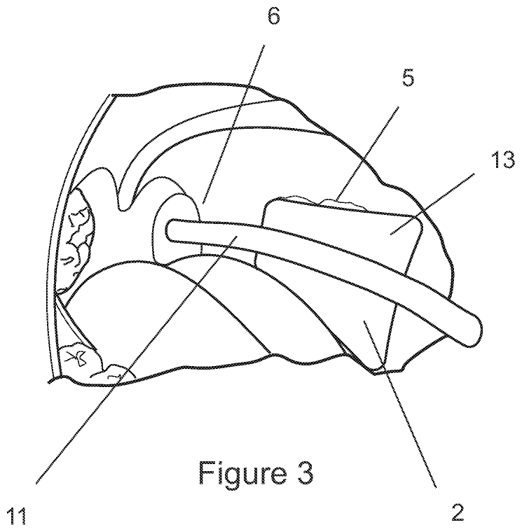

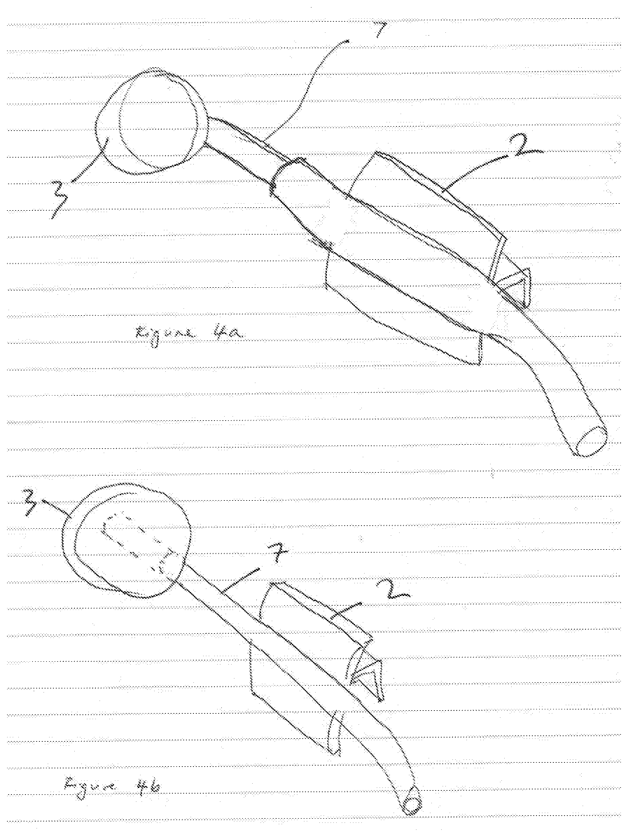

In other examples, the elongate arm 7 may be telescopic (as shown in FIG. 4), so that the length of the arm and hence the distance between the anchoring structure 2 and the wound-contacting portion 3 may be adjusted before or during positioning of the device 1 in the patient's mouth.

In some alternative configurations, the deformation may be a substantially plastic or permanent deformation. Alternatively still, one or more portions of the device 1 may additionally or alternatively be resiliently or elastically deformable.

Importantly, in these embodiments, the ability of the portion(s)/part(s) of the device to be deformed allows for the device to be customised to the size of the specific patient's anatomy, and further to maintain the constant pressure of the wound-contacting portion 3 onto the wound.

Where a portion is plastically deformable, the portion may be shaped before or during positioning of the device 1 inside the patient's mouth. It will be appreciated that to fulfil this purpose, the deformable portion may be comprise (substantially or partially) any one or more of the wound-contacting portion 3, the anchoring structure 2, the user-manipulation portion 4, the elongate arm 7, and/or any connections between these structures.

For example the anchoring portion 2 may be malleable and deformable to conform to a particular region of the patient's dentition that provides the most effective anchoring.

In the most preferred configurations, the elongate arm 7 may be shaped such that it extends substantially along only a peripheral region of the patient's mouth, e.g., substantially parallel to the alveolar process on one side of the patient's mouth. In this way, the device may be positioned substantially away from the patient's oropharynx, to increase patient comfort, improve visibility of the wounded region, and prevent/reduce the gag reflex.

It is also preferred that the device be reversible, such that it can be turned over in order that it be used on an opposite side of the patient's mouth. For some of the embodiments described in this specification, reversibility of the entire device may require the bite block to be removed, and reattached in an opposite direction for example. Alternatively, the bite block may be symmetric about at least one axis.

Further to increase patient comfort, the wound-contacting portion 3 is preferably compact. For example, the wound-contacting portion 3 may be substantially spherical, hemispherical, discoid, ovoid or cylindrical in shape. Typical tonsils are up to approximately 30 mm in size. Accordingly, it is preferred that the contacting portion will be up to approximately 50 mm, in order to cover the wound bed, which is generally larger than the excised tonsil. In addition, it is preferred in some configurations to provide a wound contacting portion three that is removable/replaceable to allow different sizes and/or be disposable.

Additionally or alternatively, the haemostatic device 1 may comprise one or more parts or portions that connect the anchoring portion 2 to the arm 7 which is/are:
resiliently deformable,
elastically deformable,
plastically deformable, or
malleable.

The deformability may be via a spring or other biasing structure, or may be due to inherent material resilience. For example, the connection between the anchoring structure 2 and the wound-contacting portion 3 may be resiliently deformable, such that pressure is applied by the wound-contacting portion 3 onto the wound.

Resilience of the deforming portion may improve the efficacy of the device, by ensuring that sufficient pressure, but not excessive pressure is applied to the wound. This may facilitate independent operation by a patient who has little experience and is under stress or in pain.

The deformation may be along one or more axis. In some configurations, the portion is deformable both vertically as well as laterally (with reference to the in use position of the device within the user's mouth). According to some configurations, the malleable portion of the body of the device may be a malleable metal. For example, polyethylene coated malleable aluminium, or any other suitable malleable material.

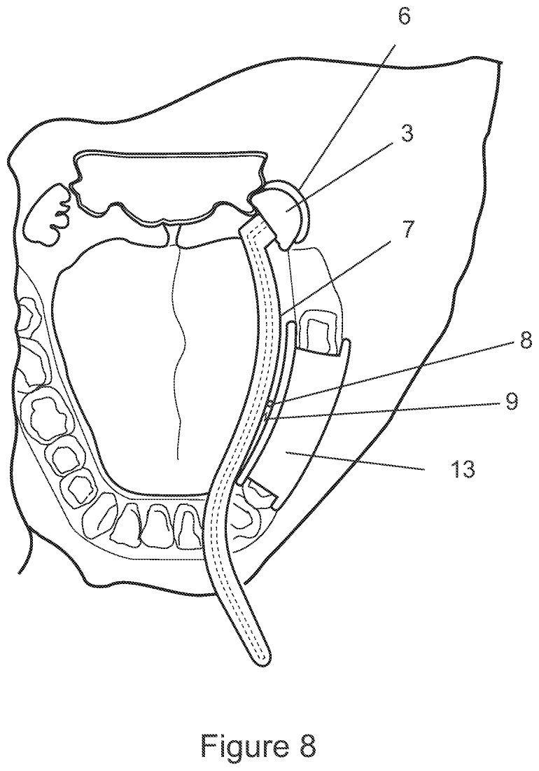

For example, with reference to FIG. 8, a resilient connection (8,9) is schematically illustrated. Portion 8 connects arm 7 and anchoring portion 2, in a movable and/or deformable manner, while biasing element 9 provides a biasing force that will act to press wound contacting portion 3 onto the tonsillar fossa 6. It will be appreciated that a number of connection designs may be suitable to satisfy this functional requirement. For example, the spring element 8 may be a deformable rubber grommet encircling a pivot type connection.

In some configurations, the hemostatic device 1 may additionally or alternatively comprise at least one portion or part which is moveable relative to one or more other portions or parts, to adjust the positioning of the wound-contacting portion 3 relative to the wound, and/or the pressure applied by the wound-contacting portion onto the wound, and/or the distance between the wound-contacting portion 3 and the user-manipulation portion 4.

In some cases, this relative movement may be substantially permanent, for example one part may be moveable relative to the other via plastic deformation as described above.

In other preferred configurations, at least one portion of the device 1 is additionally reversibly moveable relative to one or more other portions. For example, as shown in FIG. 5, the anchoring structure 2 may be slidably moveable along a length of the device, such as along the elongate arm 7 and/or along the user-manipulation portion 4. In this way, the distance between the anchoring structure 2 and the wound-contacting portion 3 may be adjusted, before and/or during insertion of the device into the patient's mouth.

In another example, parts or portions of the device 1 may be telescopically connected to each other, hence can move relative to each other. One example has been described previously in relation to FIG. 4a, in which the wound-contacting portion 3 is telescopically connected to the anchoring structure 2. In one exemplary configuration (as shown in FIG. 4b), the wound-contacting portion 3 may have a recess or channel, into and out of which the elongate arm 7 may be slid telecopically. In some configurations where a user-manipulation portion 4 is provided, the wound-contacting portion 3 may similarly be slidably or telescopically moveable along at least part of the user-manipulation portion.

In some configurations, one or more parts of the device 1 may additionally or alternatively be rotatably moveable relative to one or more other parts of the device. Such rotational movement may be about the longitudinal axis of the device (or a part of the device) and/or around a pivot axis which may be substantially transverse to the longitudinal axis of the device.

For example, the wound-contacting portion 3 and the arm 7 portion 4 may be rotate relative to each other about a pivot axis substantially perpendicular to the longitudinal axis, such that the angle between the two portions and the linear distance between the two portions may be adjustable. This allows for adjustment of the position of the wound-contacting portion 3 relative to the wound and/or the pressure applied by the wound-contacting portion onto the wound, even while the device is engaged to the patient's teeth.

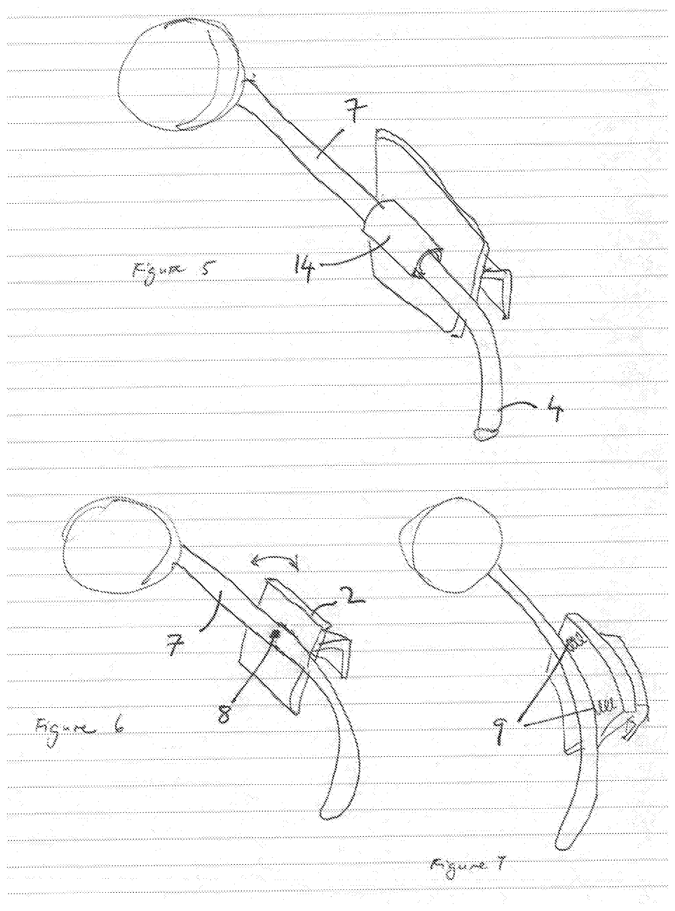

In some preferred configurations, as shown in FIG. 6, rotation is facilitated by a pivoting connection 8. For example, the wound-contacting portion may be pivotably supported on the anchoring structure 2, such that the angle between the anchoring structure and the wound-contacting portion (or the elongate arm 7) may be adjusted.

In other configurations, the relative movement between parts of the device 1 may be resilient. For example, FIG. 7 shows one embodied configuration in which some rotational movement about a pivot axis perpendicular to the longitudinal axis may be provided by a biasing connection 9 between the anchoring structure 2 and the wound-contacting portion 3.

Additionally or alternatively, the rotation may be about the longitudinal axis of the device (or a part of the device). For example, the wound-contacting portion 3 may be rotatable about a longitudinal axis of the user-manipulation portion 4 or the anchoring structure 2.

In some examples as shown in FIG. 5, the anchoring structure may be provided with a sleeve 14 which fits around the user-manipulation portion 4 or the elongate arm 7, forming a slip joint (which allows translational movement), a revolute joint (which allows rotational movement) or a cylindrical joint (which allows both rotation and translation of these components within the sleeve 14). This facilitates adjustment of the position of the wound-contacting portion 3 relative to the wound even while the device is engaged to the patient's teeth.

Alternatively, the user-manipulation portion 4 and the wound-contacting portion 3 (e.g., via elongate arm 7) may be directly kinematically coupled to each other, e.g. via a telescoping connection.

It should be understood that relative movement between one or more parts or portions of the device 1 may be provided via any suitable connection between the parts for example hinges, regions of weakness, moveable or flexible couplings, bellows, screw joints, ball joints, etc. Relative movement may additionally or alternatively be due to inherent flexibility of the material of the parts. In some configurations, it will be appreciated that the joints between the anchoring portion 2, and arm 7, may allow more than one of the above types of motion in combination.

In some configurations, the wound-contacting portion 3 comprises at least one pad 10. The pad may be made from or comprise a compressible material, such that at least a portion of the pad can conform to the shape and/or configuration of the patient's wounded anatomical feature (e.g., the surface of the patient's tonsillar fossa). The pad may alternatively or additionally be one or more of: porous, elastic, spongy, absorbent, inflatable or expandable.

In one example, the material of pad 10 is selected from one or more of: woven fabric, non-woven fabric, foam, gauze, sponge, fibre, paper, rubber, plastic, silicone or any other suitable material. Preferably, the pad 10 is hygienic, sterile or sterilisable.

According to one preferred configuration, the pad 10 may be gel filled such that it will conform to the shape of the tonsillar fossa when pressure is applied. In addition, the gel pad may be refrigerated prior to use, in order to lower rates temperature so that when the device is applied to the tonsillar fossa, it will assist vasoconstriction and/or analgesia etc.

In some configurations, the wound-contacting portion 3 may comprise one or more pre-applied substances or agents, for example: haemostatic agents, antiseptic agents, anesthetic agents, antibiotic agents, anti-inflammatory agents, cleansing agents, irrigation agents, analgesic agents, or any other medicaments that could improve the haemostasis process.

In some configurations, the agent(s) may be applied to the wound-contacting portion 3 via spraying, dipping, immersing, etc., during manufacture of the device 1 for convenience of the medical operator. In other configurations, the operator may apply the desired agent(s) to the wound-contacting portion 3 prior to inserting the wound-contacting portion into the patient's mouth.

In yet other configurations, the wound-contacting portion 3 comprises one or more reservoirs (not shown) for containing and dispensing the agent(s). The reservoir may be sealed prior to use, and the seal may be broken prior to or during insertion of the wound-contacting portion 3 into the patient's mouth to allow the agent(s) to be dispensed directly to the wound.

In some configurations, the device may additionally or alternatively comprise one or more conduits 11 that extend(s) along the length of the device. The conduit 11 may be configured to deliver, by spraying or injecting for example, one or more agents or medicaments to the wound-contacting portion or other parts of the device which are positioned in use inside the user's mouth. The conduits can allow medications to be delivered down the conduit from an external end of the device to reach the tonsillar fossa.

In further configurations, a fibre-optic channel 11 may be provided to allow a light source to be applied from the external end to assist in visualising the tonsillar fossa, and for directing insertion. If this would obviate the need for a second light source, and/or another pair of hands when positioning the device. The light source may be a bespoke light source, or alternatively could be adapted to utilise an existing device such as a pen torch or auroscope.

In addition, or alternatively still, conduit 11 may facilitate oxygen or other medical gases to be delivered to the pharynx and/or upper airway.

In a further optional configuration, the conduit may provide for a fibre-optic camera and/or cautery device to be delivered to the wound site.

In another example, the conduit may be used to deliver irrigation solution to the inside of the patient's mouth. An advantage of this conduit delivery embodiment is that medicaments/agents can be refilled and/or withdrawn from the device while it is in place inside the patient's mouth.

The conduit(s) 11 may additionally or alternatively be used to applying suction to or around the wound region and/or allow the passage of one or more medical devices, and/or provide illumination to the wound region.

In some configurations, the wound-contacting portion 3 further comprises an inflatable balloon, such that at least a portion of the wound-contacting portion 3 can be expanded to act as a tamponade for improved control of haemorrhage. The device 1 preferably further comprises means of applying pressure to inflate the balloon. In one example, pressurised gas may be supplied via conduit 11.

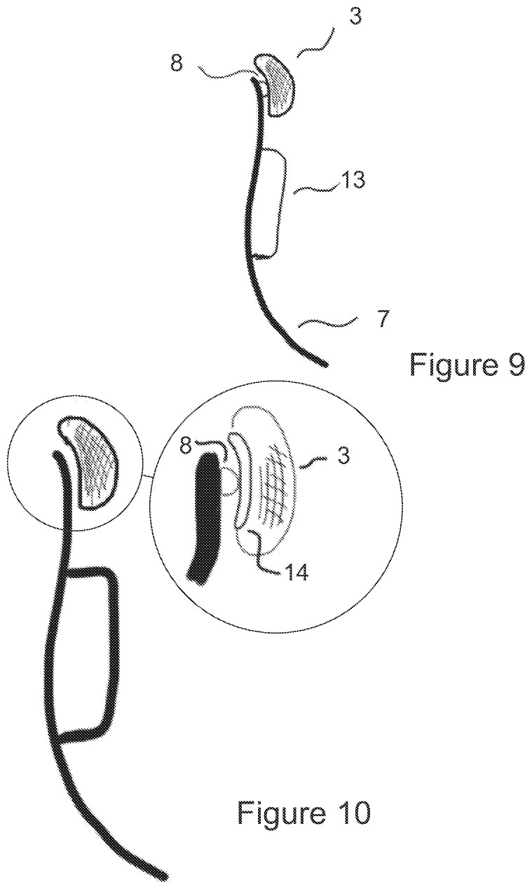

In some configurations, pad 10 may be pivotally and/or movably mounted to the end of arm 7. As shown with reference to FIG. 9, a movable joint facilitates proper alignment with the surface of the tonsillar fossa as pressure is applied.

In further alternative configurations, pad 10 may be a gauze pad or swab, or ball etc. Such configurations wouldn't necessarily require specialised pads, but rather include a gripping and/or receiving mechanism to secure a gauze pad for example. It will be appreciated that all of the described medicaments could also be used with a gauze or cotton swab, and that the gauze pad would be replaceable/changeable and disposable.

In some configurations, one or more parts of the device 1 may be detached (permanently or reversibly) from the rest of the device. In some configurations, the wound-contacting portion 3 is preferably removably attached to the arm 7. For example, with reference to FIGS. 9 & 10, the pad 10 may be removably attached.

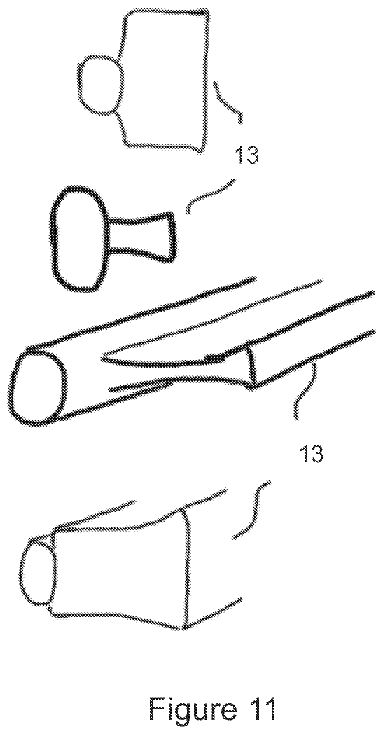

With particular reference to FIG. 10, the pad 10 may be comprised of a base plate 14 to which pad 10 is attached. The separate base plate 14 may be optionally pivotally joined to the arm 7, while a removable/replaceable pad 10 can be fitted. One advantage of a base plate, is that it provides a structure by which pressure can be applied to the wound, by the wound contacting portion. For configurations adapted to receive an easily available gauze, or cotton pad (as described above), the base plate may include a retaining mechanism for securing the wound contacting pad. Mechanisms may be a simple clip, or clips to reversibly grip the pad.

In other alternative configurations, the anchoring structure is removably attached to the device 1. Accordingly, the device may be provided as a kit of parts, such that one or more portions of the device may be replaced or substituted for another.

With particular reference to FIG. 11, a number of different bite block configurations having different size, shape and/or thicknesses etc are illustrated. It will be appreciated that varying patient anatomies may find better results with differing bite block shapes. Accordingly, it is preferred that the connection between the arm and bite block is removable to facilitate different bite block's being utilised.

The reversible attachment(s) may be via one or more suitable mechanical fasteners such clasps, threaded connections, flanges, and/or friction fit between the relevant components.

In alternative configurations, substantially the entire device (including anchoring structure 2 and wound-contacting structure 3) may be integrally formed as a unitary structure.

In some configurations, the device 1 may comprise two wound-contacting portions which are spaced from each other, and configured such that each portion contacts a patient's tonsillar fossa, on each lateral side. This may be useful to control haemorrhage from both incised regions (i.e., the left and right tonsillar fossa).

In some configurations, the second wound-contacting portion may be reversibly attached to the device 1, so that a single device may be adapted to control bleeding from the tonsillar fossa of one or both sides, depending on the patient's requirements.

Methods for managing haemorrhage using the haemostatic device will now be discussed. In some embodiments, the device 1 is first fitted or positioned into the patient's mouth by a medical operator. Subsequently, the patient handles the device themself, so that minimal supervision or intervention by the medical operator is required. In some cases, the patient may also insert and perform the initial positioning the device themself (e.g., if the device is provided as part of home care post-operative treatment).

The device 1 is positioned such that the anchoring structure 2 is engaged with the patient's teeth, for example by fitting the anchoring structure 2 onto a row of the patient's teeth. The wound-contacting portion 3 is positioned (e.g, by deforming and/or moving one or more parts of the device) to contact the patient's wound(s).

It should be appreciated that these steps may be done in any order. That is, the device may first be positioned such that the wound-contacting portion 3 contacts the wound (e.g., by deforming and/or moving one or more parts of the device, whether before the device has been inserted or while the device is in the patient's mouth). Once the wound-contacting portion 3 is in the correct position, the anchoring structure 2 may be engaged with the patient's teeth, e.g., if a bite block 13 is provided, the patient bites down on the bite block.

Subsequent adjustment of the position of the wound-contacting portion 3 may be necessary to ensure effective pressure is applied on the wound. The position and pressure applied by the wound-contacting portion 3 may be adjusted by moving or deforming one or several parts of the device, depending on which embodiment of the device is employed. For example, where the wound-contacting portion 3 is telescopically connected to the user-manipulation portion 4, the telescopic connection may be extended or retracted as necessary to move the wound-contacting portion further into or out of the patient's throat region. Suitable methods for adapting other embodiments of the device should be apparent from the description of the different embodiments above.

Once in place, the patient may independently operate and manipulate the device, so that intervention by the medical operator may not be required during most of the duration of the treatment. In some cases, the device is shaped to fit the patient's anatomy (whether during manufacture, or before or during insertion into the patient's mouth), so that little more is needed than to maintain the position of the device. The anchoring structure 2 may provide sufficient securement of the device 1 to the patient. Additionally, if a user-manipulation portion 4 is provided as a handle, the patient may also grip the handle to help stabilise and maintain the position and pressure of the wound-contacting portion 3 on the wound.

In other configurations, the patient or a medical operator may periodically manipulate the device 1, e.g., via user-manipulation portion 4, to adjust the position of the wound-contacting portion 3 or the pressure applied by the wound-contacting portion on the wound, as required for effective treatment.

Once the treatment is completed or suspended (e.g., for further surgical intervention), the device may be removed from the patient's mouth by simply disengaging the anchoring structure from the patient's teeth.

The foregoing description of the invention includes preferred forms thereof. Modifications may be made thereto without departing from the scope of the invention as defined by the accompanying claims.

1. A haemostatic device comprising:
at least one anchoring structure configured to be reversibly engaged with a patient's teeth,
an arm connected to said anchoring structure, and
at least one wound-contacting portion at or toward an end of the arm.

2. The haemostatic device of claim 1, further comprising a user-manipulation portion at or toward the other end of said arm, said user-manipulation portion being manipulatable to adjust:
positioning of said wound-contacting portion, and
pressure applied by said wound-contacting portion(s) onto one or more wound(s).

3. The haemostatic device of claim 1 wherein the anchoring structure comprises at least one bite block to be held between the patient's mandibular and maxillary teeth.

4. The haemostatic device of any one of the preceding claims, claim 1 wherein said wound-contacting portion is supported from said anchoring structure via the elongate arm.

5. The haemostatic device of claim 4, wherein said elongate arm is integrally formed with said user-manipulation portion.

6. The haemostatic device of claim 1 wherein said connection between said arm and said anchoring structure is movable, to adjust one or more of:
positioning of said wound-contacting portion,
pressure applied by said wound-contacting portion onto said wound, shape and configuration of said wound-contacting portion, distance between said wound-contacting portion and said user-manipulation portion.

7. The haemostatic device of claim 6, wherein the movable connection is resiliently deformable via one or more of:
a spring or other biasing structure,
inherent resilience of the material of said portion.

8. The haemostatic device of claim 6, wherein said moveable connection is plastically deformable, such that said portion may be shaped prior to application of said wound-contacting portion on said wound.

9. (canceled)

10. The haemostatic device of any one of claim 6 wherein a connection between said wound-contacting portion and said arm is moveable or deformable.

11. and 12. (canceled)

13. The haemostatic device of claim 2 comprising at least one portion which is movable relative to one or more other portions, to adjust one or more of:
positioning of said wound-contacting portion,
pressure applied by said wound-contacting portion onto said wound,
distance between said wound-contacting portion and said user-manipulation portion.

14. (canceled)

15. The haemostatic device of claim 13, wherein said anchoring structure is slidably movable along said arm.

16. The haemostatic device of claim 13 wherein said anchoring structure is rotatably movable relative to said arm.

17. (canceled)

18. The haemostatic device of claims 13, wherein said arm is pivotably supported on said anchoring structure.

19. The haemostatic device of claim 13 wherein said wound-contacting portion is slidably or telescopically movable along the arm.

20. The haemostatic device of claim 1 wherein the wound-contacting portion comprises a pad, and said pad comprises one or more of:
a. woven fabric,
b. non-woven fabric,
c. foam,
d. gauze,
e. sponge,
f. fibre,
g. paper,
h. a gel block, or a gel filled capsule,
i. plastic, or
j. rubber.

21.-23. (canceled)

24. The haemostatic device of claim 20 wherein said wound-contacting portion comprises one or more pre-applied:
a. Haemostatic agent,
b. Antiseptic agent,
c. Anesthetic agent,
d. Antibiotic agent,
e. Anti-inflammatory agent,
f. Cleansing agent,
g. Irrigation agent,
h. Analgesic agent,
said one or more agents deliverable to said wound when said wound-contacting portion is applied to said wound.

25. (canceled)

26. The haemostatic device of claim 1 wherein said wound-contacting portion comprises an inflatable balloon, and wherein said device further comprising means of applying a pressure to inflate said balloon.

27.-33. (canceled)

34. The haemostatic device of claim 1 wherein said anchoring structure comprises a channel sized and shaped to fit over a patient's teeth.

35. The haemostatic device of claim 1 wherein said anchoring structure includes a deformable/mouldable material, to improve and/or maximise dental contact.

36.-37. (canceled)

38. A method of controlling bleeding of a patient from one or more wound(s) using the haemostatic device of claim 1 comprising:
a. engaging said anchoring structure to the patient's teeth,
b. positioning said wound-contacting portion on said wound.

39. A method of controlling bleeding of a patient from one or more wound(s) using the haemostatic device of claim 38 further comprising a step of manipulating said user-manipulation portion to adjust the pressure applied by said wound-contacting portion onto said wound.

40. The method of claim 39, wherein the patient manipulates said user-manipulation portion to adjust one or both of:
a. position of said wound-contacting portion,
b. pressure applied by said wound-contacting portion onto said wound.
